(19) United States
(12) Patent Application Publication
(10) Pub. No.: US 2023/0348993 A1
(43) Pub. Date: Nov. 2, 2023

(54) DIAGNOSIS OF CANCER OR OTHER PHYSIOLOGICAL CONDITION USING CIRCULATING NUCLEIC ACID FRAGMENT SENTINEL ENDPOINTS

(71) Applicant: BELLWETHER BIO, INC., REEDWOOD CITY, CA (US)

(72) Inventors: Matthew William SNYDER, Seattle, WA (US); Robert Navid Farjad AZAD, Seattle, WA (US); Jay SHENDURE, Seattle, WA (US)

(73) Assignee: BELLWETHER BIO, INC., REDWOOD CITY, CA (US)

(21) Appl. No.: 18/139,231

(22) Filed: Apr. 25, 2023

Related U.S. Application Data
(63) Continuation of application No. 16/705,769, filed on Dec. 6, 2019, now abandoned.

(51) Int. Cl. C12Q 1/6886; G16H 50/20; G16B 35/10; C12Q 1/6874; C40B 30/00; G16B 20/00

(52) U.S. Cl. CPC C12Q 1/6886; G16H 50/20; G16B 35/10; C12Q 1/6874; C40B 30/00; G16B 20/00; G16B 30/10

(57) ABSTRACT

Methods for diagnosis of cancer or other physiological conditions using cDNA as sentinel endpoints are disclosed.

### RELATED APPLICATION

This application is a continuation of U.S. patent application Ser. No. 16/705,769, filed Dec. 6, 2019 which is a 35 U.S.C. 371 National Stage of International Application No. PCT/US2018/036963, filed Jun. 11, 2018, which claims priority to and the benefit of U.S. Provisional Application No. 62/517,571, filed Jun. 9, 2017, the contents of which are hereby incorporated by reference herein in their entirety.

### FIELD

Provided are methods for diagnosis of one or more physiological conditions using cfDNAs to construct sentinel endpoints.

### BACKGROUND

Cell-free DNA (cfDNA) is present in the circulating plasma, urine, and other bodily fluids of humans. cfDNA contains both single and double stranded DNA fragments that are relatively short and are normally found at low concentrations in plasma. In the circulating plasma of healthy individuals, cfDNA is believed to derive from apoptosis of blood cells. However, other tissues can contribute to cfDNA in plasma.

In recent years, efforts have been made to exploit cfDNA in conjunction with the emergence of new technologies related to cost-effective DNA sequencing in the development of diagnostics. In pregnant women, for example, a proportion of cfDNA in circulating plasma derives from fetal or placental cells. Screening for genetic abnormalities in the fetus, such as chromosomal trisomies, can be achieved by deep sequencing of the cfDNA of a pregnant woman, since the cfDNA of a pregnant woman is a mixture of cfDNA derived from the maternal and fetal genomes. One can expect to observe an excess of reads mapping to chromosome 21 if the fetus has trisomy 21. Non-invasive screening based on analysis of cfDNA is now routinely offered to pregnant women.

With respect to cancer diagnostics, a proportion of cfDNA in circulating plasma can come from a tumor, with the contribution from the tumor often increasing with cancer stage. Cancer is caused by abnormal cells exhibiting uncontrolled proliferation secondary to mutations in their genomes. The observation of mutations in cfDNA has substantial promise to effectively serve as a diagnostic for cancer.

With respect to transplant rejection, after a transplant is performed, there is a risk of allogruft rejection. Currently, the gold standard for assessing transplant rejection involves an invasive biopsy. A major challenge is determining whether and to what extent a rejection is occurring without an invasive biopsy. Recently, using cfDNA from the donor as a non-invasive marker for detecting allograft rejection has been explored.

There are several shared characteristics of current cfDNA diagnostic efforts. First, each relies on sequencing of cfDNA, generally from circulating plasma but potentially from other bodily fluids. Second, each relies on the fact that cfDNA comes from cell populations bearing genomes that differ very little from one another with respect to primary nucleotide sequence and/or copy number. Third, the basis for each is to detect or monitor genotypic differences between cell populations.

The reliance of cfDNA efforts in diagnostics on what are essentially genotypic differences is the basis of their success but also a major limitation. For example, since an overwhelming majority of cfDNA corresponds to regions of the human genome that are identical, the reliance on genotypic differences is uninformative when one is trying to discriminate between cell populations or between one group of subjects and another.

There is a need for a cfDNA test with greater discriminatory power.

### SUMMARY

Provided herein are methods for using cfDNA to discriminate between groups using sentinel endpoints. Sentinel endpoints comprise genomic coordinates that are far from a defined mathematical function. The defined mathematical function is an equation that segregates distributions of quantities of cfDNA fragment endpoints from linked groups comprising the number of fragment endpoints observed at a genomic location.

Also provided herein are methods for diagnosing a disease using sentinel endpoints. In some embodiments, a subject is diagnosed as having a disease if the number of sentinel endpoints is above a threshold value.

In one aspect, the invention is drawn to a method of identifying one or more sentinel endpoints comprising:
a. isolating cfDNA from biological sample(s) from one or more subjects with at least one first physiological state, the isolated cfDNA comprising a first plurality of cfDNA fragments;
b. constructing at least one first sequencing library from the first plurality of cfDNA fragments;
c. sequencing first fragment endpoints of the first plurality of cfDNA fragments;
d. determining genomic locations of the first fragment endpoints within a reference genome for at least some of the first plurality of cfDNA fragments as a function of the sequences;
e. determining a first vector comprising the number of first fragment endpoints observed at each genomic location;
f. isolating cfDNA from biological sample(s) from one or more subjects with at least one second physiological state, the isolated cfDNA comprising a second plurality of cfDNA fragments;
g. constructing at least one second sequencing library from the second plurality of cfDNA fragments;
h. sequencing second fragment endpoints of the second plurality of cfDNA fragments;
i. determining genomic locations of the second fragment endpoints within the reference genome for at least some of the second plurality of cfDNA fragments as a function of the sequences;
j. determining a second vector comprising the number of second fragment endpoints observed at each genomic location;
k. linking the first vector and the second vector;
l. defining a mathematical function to segregate distributions of quantities from the linked vectors into a first group and a second group, the first group comprising genomic coordinates with lesser difference of quantities to the mathematical function and the second group comprising genomic coordinates with greater difference of quantities to the mathematical function; and
m. identifying one or more sentinel endpoints as members of the second group. In some embodiments, the greater difference of quantities comprises or consists of about 10%, about 20%, about 30%, about 40%, about 50%, about 60%, about 70%, about 80%, about 90%, or about 95% greater difference of quantities between the second group and the mathematical function. In some embodiments, the lesser difference of quantities to the mathematical function comprises or consists of about 10%, about 20%, about 30%, about 40%, about 50%, about 60%, about 70%, about 80%, about 90%, or about 95% lesser difference of quantities between the first group and the mathematical function.

In some embodiments, the method further comprises diagnosing a disease or physiological condition in a subject in need thereof, wherein the at least one first physiological state is a healthy state and the at least one second physiological state is a disease state, comprising:
a. isolating cfDNA from biological sample(s) from the subject, the isolated cfDNA comprising a subject plurality of cfDNA fragments;
b. constructing a subject sequencing library from the subject plurality of cfDNA fragments;
c. sequencing subject fragment endpoints of the subject plurality of cfDNA fragments;
d. determining genomic locations of at least some of the subject fragment endpoints within the reference genome as a function of the sequences;
e. determining a subject vector comprising the number of subject fragment endpoints observed at each genomic location; and
f. diagnosing the disease or physiological condition in the subject if the number of sentinel endpoints in the human subject vector is above a threshold value.

In some embodiments, at least some of the isolated cfDNA are filtered to retain cfDNA having a length between. an. upper bound and a lower bound. In some embodiments, the upper bound is 200, 190, 180, 170, 160, 150, 140, 130, 120, 110, 100, 90, 80, 70, 60, or 50 base pairs and the lower bound is 20, 25, 30, 35, 36, 40, 45, 50, 60, 70, 80, 90, 100, 110, or 120 base pairs.

In some embodiments, a subset of any of the isolated cfDNA is targeted to a genomic location. In sonic embodiments, the genomic location comprises one or more genomic annotations. In some embodiments, the method comprises filtering sentinel endpoints based upon proximity to one or more genomic annotations.

In some embodiments, the one or more genomic annotations comprises DNA-binding or DNA-contacting proteins, in some embodiments, the one or more genomic annotations comprises or consists of transcription start sites (TSSs). In some embodiments, the one or more genomic annotations comprises or consists of nucleosomes.

In some embodiments, the method further comprises providing a report with scores. In some embodiments, the method further comprises recommending treatment for the diagnosed disease or physiological condition in the subject.

In some embodiments, the disease or physiological condition, at least one first physiological state, or at least one second physiological state is selected from the group consisting of cancer, normal pregnancy, complications of pregnancy, myocardial infarction, inflammatory bowel disease, systemic autoimmune disease, localized autoimmune disease, allotransplantation with rejection, allotransplantation without rejection, stroke, and localized tissue damage. In some embodiments, the disease comprises cancer. In some embodiments, the cancer is colorectal cancer or ovarian cancer. In some embodiments, the disease or physiological condition, at least one first physiological state, or at least one second physiological state is a healthy state.

In some embodiments, the biological sample comprises or consists of whole blood, peripheral blood plasma, urine, or cerebral spinal fluid.

In some embodiments, the method further comprises determining that a disease or physiological condition in a subject has an increased burden, severity, or clinical stage, wherein the at least one first physiological state is a disease state or physiological condition and the at least one second physiological state is the disease state or physiological condition with an increased burden, severity, or clinical stage, the method comprising:
a. isolating cfDNA from biological sample(s) from the subject, the isolated cfDNA comprising a subject plurality of cfDNA fragments;
b. constructing a subject sequencing library from the subject plurality of cfDNA fragments;
c. sequencing subject fragment endpoints of the subject plurality of cfDNA fragments;
d. determining genomic locations of the subject fragment endpoints within the reference genome for at least some of the subject plurality of cfDNA fragments as a function of the sequences;
e. determining a subject vector comprising the number of subject fragment endpoints observed at each genomic location;
f. comparing the subject vector to the sentinel endpoints;
g. identifying the burden, severity, or clinical stage of the disease or physiological condition as having an increased burden, severity, or clinical stage if the subject vector has more sentinel endpoints than a threshold value. In some embodiments, the at least one first physiological state consists of a cancer at stage 0, stage I, stage II, stage III, or stage IV. In some embodiments, the at least one second physiological state consists of a cancer at stage 0, stage I, stage II, stage III, or stage IV.

### BRIEF DESCRIPTION OF THE FIGURES

FIG. 1 depicts an example of a binary count visualization.

FIG. 2 depicts an example of a binary count visualization with a single thumb.

FIG. 3 depicts frequency of endpoint coordination in healthy and colorectal cancer (“CRC”) samples.

FIG. 4 depicts frequency of endpoint coordination in healthy and ovarian cancer (“OVC”) samples.

FIG. 5 depicts the number of sequencing datasets in which an autosomal genomic coordinate was observed at least once as a fragment endpoint for healthy and CRC samples.

FIG. 6 depicts the number of fragments that appeared as an endpoint for each autosomal genomic coordinate for healthy and CRC samples.

FIG. 7A and FIG. 7B depict the number of sentinel endpoints at each of a set of 1039 sentinel endpoint coordinates for healthy and CRC samples. Distributions for healthy and CRC samples are shown in a histogram in FIG. 7A. Distributions for healthy and CRC are shown in a boxplot in FIG. 7B.

FIG. 8 depicts the performance of a binary classifier used to assign a label of healthy and disease based on the number of sentinel endpoint observations.

### DESCRIPTION OF EXEMPLARY EMBODIMENTS

The present invention provides methods for using cfDNA to discriminate between groups using sentinel endpoints. Sentinel endpoints comprise genomic coordinates that are far from a defined mathematical function. The defined mathematical function is an. equation that segregates distributions of quantities of cfDNA fragment endpoints from linked groups comprising the number of fragment endpoints observed at a genomic location.

The present invention also provides methods for diagnosing a subject as having a disc se using sentinel endpoints. In some embodiments, a subject is diagnosed as having a disease it the number of sentinel endpoints is above a threshold value.

### I. Definitions

As used herein. “allotransplantation” refers to the transplantation of tissues, or organs to a recipient from a genetically non-identical donor of the same species. The transplant is called an allograft, allogeneic transplant, or homograft. Most human tissue and organ transplants are allografts.

As used herein, “annotations,” “DNA annotations,” “genome annotation,” “genomic annotations” refer to the locations of genes, coding regions, and functional areas and the determination of what those genes, coding regions, and functional areas do.

As used herein, “autoimmune disease” refers to a condition resulting from an abnormal immune response to a normal body part.

As used herein, “binary counts” refers to fragment endpoint counts that each dataset contributes a one or a zero at each genomic coordinate.

As used herein, “burden” refers to a load or weight with respect to a particular disease or physiological condition such as, for example, an increased stage in cancer. In some embodiments, sentinel endpoints may be used to determine an increased burden.

As used herein, “cancer” refers to disease caused by an uncontrolled division of abnormal cells in a part of the body.

As used herein, “cell-free DNA” or “cfDNA” refers to DNA fragments present in the blood plasma.

As used herein. “fragment endpoints” or “endpoints” shall refer to the termini. of cfDNA.

As used herein, “genome” or “genomic” refers to the complete set of genes or genetic material present in a cell or organism.

As used herein, “integer counts” refers to fragment endpoint counts such that each dataset contributes to the fragment endpoint count at each coordinate based on the number of times that coordinate appears as a fragment endpoint.

As used herein, “inflammatory bowel disease” refers to group of chronic intestinal diseases characterized by inflammation of the bowel in the large or small intestine. The most common types of inflammatory bowel disease are ulcerative colitis and Crohn's disease.

As used herein, “myocardial infarction” refers to the irreversible death or necrosis of heart muscle secondary to prolonged lack of oxygen supply,

As used herein, “next generation sequencing” refers to any high-throughput sequencing approach including, but not limited to, one or more of the following: massively-parallel signature sequencing, pyrosequencing (e.g., using a Roche 454 sequencing device), Illumina sequencing, sequencing by synthesis, ion torrent sequencing, sequencing by ligation. (“SOLiD”), single molecule real-time (“SMRT”) sequencing, colony sequencing, DNA nanoball sequencing, heliscope single molecule sequencing, and nanopore sequencing.

As used herein, “peripheral blood” refers to the flowing, circulating blood of the body. It is normally composed of erythrocytes, leukocytes, and thrombocytes. These blood cells are suspended in blood plasma, through which the blood cells are circulated through the body. Peripheral blood is different from blood whose circulation is enclosed within the liver, spleen, bone marrow, and the lymphatic system. These areas contain their own specialized blood.

As used herein, “peripheral blood plasma” refers to the plasma found in peripheral blood.

As used herein, “plasma” or “blood plasma” refers to the liquid component of blood that normally holds the blood cells in whole blood in suspension. Holding blood cells in whole blood makes plasma the extracellular matrix of blood cells.

As used herein, “proximity” refers to nearness in space or relationship. In some embodiments, proximity refers to nearness of genomic coordinates as orientated on a reference genome. In some embodiments, proximity refers to the nearness of fragment endpoints, one to another. In some embodiments, proximity refers to nearness of sentinel endpoints to each other as members of a group. In some embodiments, proximity refers to nearness of fragment endpoints as members of a group.

As used herein, “sentinel endpoints” refers to genomic coordinates that appear as termini of cfDNA fragments more frequently in one state as opposed to another state.

As used herein, “stroke” refers to the sudden death of brain cells due to lack of oxygen caused by blockage of blood flow or rupture or an artery to the brain.

As used herein, “threshold value” refers to a value greater than an empirically determined number of sentinel endpoints.

As used herein, “vector” shall refer to points arising from the number of fragment endpoints observed at each genomic location. In mathematics, a vector is conceived as an object that has both a magnitude and a direction. A vector, as used herein, then, has a magnitude of the number of fragment endpoints at a given location and a direction determined with respect to genomic location.

As used herein, “whole blood” refers to blood drawn directly from the body from which no components, such as plasma or platelets, have been removed.

### II. Subjects

A subject may be any subject known to one skilled in the art. In some embodiments, the subject is human. In some embodiments, the subject is non-human. A human subject can be any gender, such as male or female. In some embodiments, the human can be an infant, child, teenager, adult, or elderly person. In some embodiments, the subject is a female subject who is pregnant, suspected of being pregnant, or planning to become pregnant.

In some embodiments, the subject is a mammal, a non-human mammal, a non-human primate, a primate, a domesticated animal (e.g., laboratory animals, household pets, or livestock), or a non-domesticated animals (e.g., wildlife). In some embodiments, the subject is a dog, cat, rodent, mouse, hamster, cow, bird, chicken, pig, horse, goat, sheep, rabbit, ape, monkey, or chimpanzee.

### III. Biological Samples

Biological samples can be any type known to one skilled in the art and may be obtained from any subject. In some embodiments, the biological sample is from a human subject. In some embodiments, the biological sample is from a non-human subject. In some embodiments, a biological sample is isolated from one or more subjects having one or more physiological states. In some embodiments, the one or more physiological states are one or more healthy or disease states. In some embodiments, the one or more physiological states are one or more healthy human states and/or human disease states.

in some embodiments, biological samples comprise or consist of unprocessed samples (e.g., whole blood, tissue, or cells) or processed samples (e.g., serum or plasma). In some embodiments, biological samples are enriched for a certain type of nucleic acid. In some embodiments, biological samples are processed to isolate nucleic acids from other components within the biological sample.

In some embodiments, biological samples comprise cells, tissue, a bodily fluid, or a combination thereof in some embodiments, biological samples comprise or consist of whole blood, peripheral blood plasma, urine, or cerebral spinal fluid. In sonic embodiments, biological samples comprise or consist of a blood components, plasma, serum, synovial fluid, bronchial-alveolar lavage, saliva, lymph, spinal fluid, nasal swab, respiratory secretions, stool, peptic fluids, vaginal fluid, semen, and/or menses.

In some embodiments, biological samples comprise or consist of fresh samples. In some embodiments, biological samples comprise or consist of frozen samples. In some embodiments, biological samples comprise or consist of fixed samples, e.g., samples fixed with a chemical fixative such as formalin-fixed paraffin-embedded tissue.

Biological samples may also be obtained at any point during medical care. In some embodiments, biological samples are obtained prior to treatment, during the treatment process, after diagnosis, or any other point. Biological samples may be obtained at specific internals, such as weekly or monthly or during routine medical examinations.

Isolating cfDNA

isolation of cfDNA can proceed according to any method known to one skilled in the art. For example, the QIAGEN QlAamp Circulating Nucleic Acid kit is commonly used to isolate cfDNA from plasma or urine based on the binding of cfDNA to a silica column. An alternative method, phenol-chloroform extraction followed by isopropanol or ethanol precipitation, provides similar results.

In some embodiments, isolating cfDNA is done in such a manner as to maximize the recovery of short fragments (<100 base pairs), as the composition of short fragments differs more strongly between healthy and disease states than the composition of longer fragments does between healthy and disease samples. In some embodiments, any of the cfDNA fragments are subjected to a size selection to retain only cfDNA fragments having a length between an upper bound and a lower bound. In some embodiments, the upper bound is 200, 190, 180, 170, 160, 150, 140, 130, 120, 110, 100, 90, 80, 70, 60, or 50 base pairs and the lower bound is 20, 25, 30. 35, 36, 40, 45, 50, 60, 70, 80, 90, 100, 110, or 120 base pairs. In some embodiments, only the lower bound is 36 and the upper bound is 100.

### V. Constructing a Sequencing Library

After isolating cfDNA from a biological sample, isolated cfDNA comprising a plurality of cfDNA fragments can be subjected to one or more enzymatic steps to create a sequencing library. Enzymatic steps can proceed according to techniques known to those of skill in the art. Enzymatic steps may include 5′ phosphorylation, end repair with a polymerase, A-tailing with a polymerase, ligation of one or more sequencing adapters with a ligase, and linear or exponential amplification of with a polymerase.

Preparation of sequencing libraries may be performed to maximize the conversion of short fragments (<100 base pairs). In some embodiments, a physical size-selection step is employed to select for short cfDNA fragments. In some embodiments, an enrichment step is employed, wherein the enrichment step comprises enriching cfDNA that are targeted to a genomic location. An enrichment step may be employed by itself or in conjunction with a physical size-selection step. A physical size selection step could comprise or consist of gel electrophoresis and/or capillary electrophoresis. In some embodiments, constructing a sequencing library should preserve the original termini of cfDNA fragments.

Some embodiments comprise attaching adapters to the plurality of cfDNA fragments to aid in purification, detection, amplification, or a combination thereof. In some embodiments, the adapters are sequencing adapters. In some embodiments, at least some of the plurality of cfDNA fragments are attached to the same adapter. In some embodiments, different adaptors are attached at both ends of the plurality of cfDNA fragments. In some embodiments, at least some of the plurality of cfDNA fragments may be attached to one or more adapters on one end. Adapters may be attached to cfDNAs by primer extension, reverse transcription, or hybridization.

In some embodiments, an adapter is attached to a plurality of cfDNA fragments by ligation. In some embodiments, an adapter is attached to a plurality of cfDNA fragments by a ligase. In some embodiments, an adapter is attached to a plurality of cfDNA fragments by sticky-end ligation or blunt-end ligation. An adapter may be attached to the 3′ end, the 5′ end, or both ends of the plurality of cfDNA fragments.

In some embodiments, enzymatic end-repair processes are used for adapter ligation. The end repair reaction may be performed by using one or more end repair enzymes (e.g., a polymerase and an exonuclease).

In some embodiments, the ends of the plurality of cfDNA fragments can be polished by treatment with a polymerase. Polishing can involve removal of 3′ overhangs, fill-in of 5′ overhangs, or a combination thereof For example, a polymerase may fill in the missing bases for a DNA strand from 5′ to 3′ direction. The polymerase can be a proofreading polymerase (e.g., comprising 3′ to 5′ exonuclease activity). The proofreading polymerase can be, e.g., a T4 DNA polymerase, Pol 1 Klenow fragment, or Pfu polymerase. Polishing can comprise removal of damaged nucleotides using any means known in the art. In some embodiments, the ends of the plurality of cfDNA fragments are polished by treatment with an exonuclease to remove the 3′ overhangs.

### VI. Sequencing of Fragment Endpoints

In some embodiments, sequencing fragment endpoints of the plurality of cfDNA fragments comprises or consists of sequencing the plurality of cfDNA fragments. In some embodiments, sequencing fragment endpoints of the plurality of cfDNA fragments comprises or consists of sequencing entire cfDNA fragments) of the plurality of cfDNA fragments. In some embodiments, sequencing fragment endpoints of the plurality of cfDNA fragments comprises or consists of sequencing only the fragment endpoints of the plurality of cfDNA fragments.

Following the preparation of a sequencing library, at least the fragment endpoints of the plurality of cfDNA fragments are sequenced. Any method known to one skilled in the art may be used to generate a dataset consisting of at least one “read” (the ordered list of nucleotides comprising each sequenced molecule). In some embodiments, sequencing fragment endpoints comprises or consists of next generation sequencing assay.

In some embodiments, sequencing comprises or consists of classic Sanger sequencing, methods that are well known in the art. In some embodiments, sequencing comprises or consists of sequencing on an Illumina Novaseq instrument with an S4 flow cell. In some embodiments, sequencing comprises or consists of sequencing on Illumina's Genome Analyzer IIX, MiSeq personal sequencer, NextSeq series, or HiSeq systems, such as those using HiSeq 4000, HiSeq 3000, HiSeq 2500, HiSeq 1500, HiSeq 2000, or HiSeq 1000. In some embodiments, sequencing comprises or consists of using technology available by 454 Lifesciences, Inc. to sequence fragment endpoints. In some embodiments, sequencing comprises or consists of ion semiconductor sequencing (e.g., using technology from Life Technologies (Ion Torrent)).

In some embodiments, sequencing comprises or consists of nanopore sequencing (See e.g., Soni G V and Meller A. (2007) Clin Chem 53: 1996-2001, which is incorporated by reference in its entirety, including any drawings). In some embodiments, nanopore sequencing comprises or consists of using technology from Oxford Nanopore Technologies; e.g., a GridION system. In some embodiments, nanopore sequencing comprises or consists of strand sequencing in which intact DNA polymers can be passed through a protein nanopore with sequencing in real time as the DNA translocates the pore.

In some embodiments, nanopore sequencing comprises or consists of exonuclease sequencing in which individual nucleotides can be cleaved from a DNA strand by a processive exonuclease and the nucleotides can be passed through a protein nanopore. In some embodiments, nanopore sequencing comprises or consists of nanopore sequencing technology from GENIA. In some embodiments, nanopore sequencing comprises or consists of technology from NABsys. In some embodiments, nanopore sequencing comprises or consists of technology from IBM/Roche.

In some embodiments, sequencing comprises or consists of sequencing by ligation approach. One example is the next generation sequencing method of SOLiD sequencing. SOLID may generate hundreds of millions to billions of small sequence reads at one time.

### VII. Determining Genomic Locations of Fragment Endpoints

For each dataset (i.e., for each sequenced library of a plurality of fragment endpoints), the two genomic endpoints of each sequenced fragment endpoints are extracted with computer software. Alter sequencing of cfDNA fragments and fragment endpoints and appropriate quality control, a gnomic location for the fragment endpoints within a reference genome is determined. The process of determining genomic locations, or mapping, identities the genomic origin of each fragment based on a sequence comparison, determining, for example, that a given fragment of cfDNA was originally part of a specific region of chromosome 12. Determining genomic locations of fragment endpoints can be done with any human reference genome, such as, for example. Genbank hg19 or Genbank hg38, using bwa software (See, http://bio-bwa.sourceform.net/, which is incorporated by reference herein; See. WO 2016/015058, which is incorporated by reference herein in its entirety, including any drawings.).

The procedure is performed for each library derived from each biological sample to produce one dataset per library. The procedure of mapping provides two fragment endpoints for each cfDNA fragment The fragment endpoints are given numerical values (“coordinates”), representing the specific offset, relative to one end of a chromosome, of the fragment endpoint's location within the reference genome.

In some embodiments, fragment endpoints are further oriented in two dimensions, such that for every fragment endpoint, a given fragment endpoint's coordinate is either greater than or less than its partner's coordinate, in other words, each fragment endpoint is the left-most or right-most fragment endpoint coordinate of the pair in two-dimensional space. In some embodiments, a plurality of the fragment endpoints are classified based on the strand, for example Watson or Crick, from which their associated, sequenced cfDNA fragment was derived.

In some embodiments, the genomic location of the first fragment endpoints and the second reference fragment endpoints may be determined with an available database. In some embodiments, the available database comprises or consists of a public database.

Accordingly, when using an available database, the invention is drawn to a method of identifying one or more sentinel endpoints comprising:
a. using an available database of fragment endpoints, determining genomic locations of first fragment endpoints of at least one first physiological state within a reference genome with the available database;
b. determining a first vector comprising the number of first fragment endpoints observed at each genomic location;
c. using an available database of fragment endpoints, determining genomic locations of second fragment endpoints of at least one second physiological state within a reference genome with the available database;
d. determining a second vector comprising the number of second fragment endpoints observed at each genomic location;
e. linking the first vector and the second vector;
f. defining a mathematical function to segregate distributions of quantities from the linked vectors into a first group and a second group, the first group comprising genomic coordinates with lesser difference to the mathematical function and the second group comprising genomic coordinates with greater difference to the mathematical function; and
g. identifying one or more sentinel endpoints as members of the second group.

### VIII. Determining a Vector

Vectors are determined with the number of fragment endpoints observed at each genomic location. Some embodiments comprise a set of two or more vectors, each having a single entry for a single coordinate under consideration. In some embodiments, the set of two or more vectors comprise or consist of one vector of binary counts for each physiological state and one vector of integer counts for the same respective physiological states. In some embodiments, for example, the physiological states comprise a healthy state. In some embodiments, the physiological states comprise a disease state.

in some embodiments, the set of two or more vectors are visualized. In some embodiments, the set of two of more vectors are visualised as a two-dimensional histogram or scatterplot, where one axis indicates either binary or integer counts for a disease state and the other axis represents binary or integer counts for the another disease state.

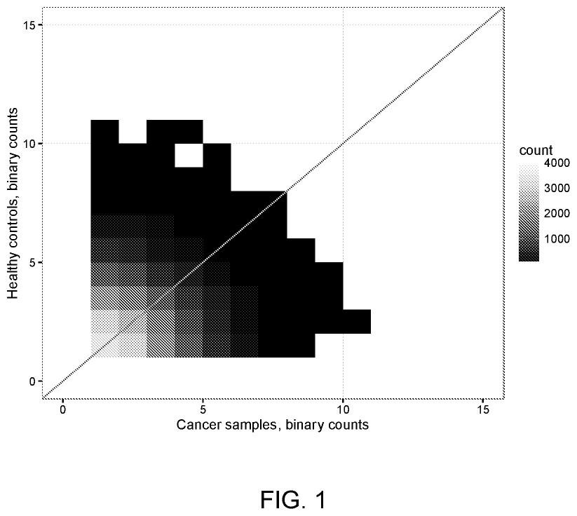

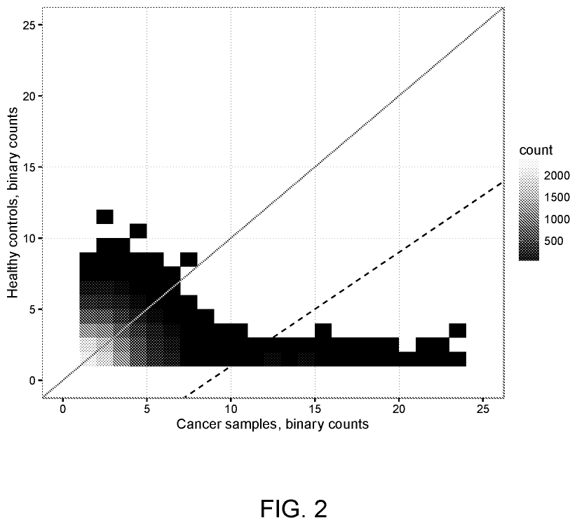

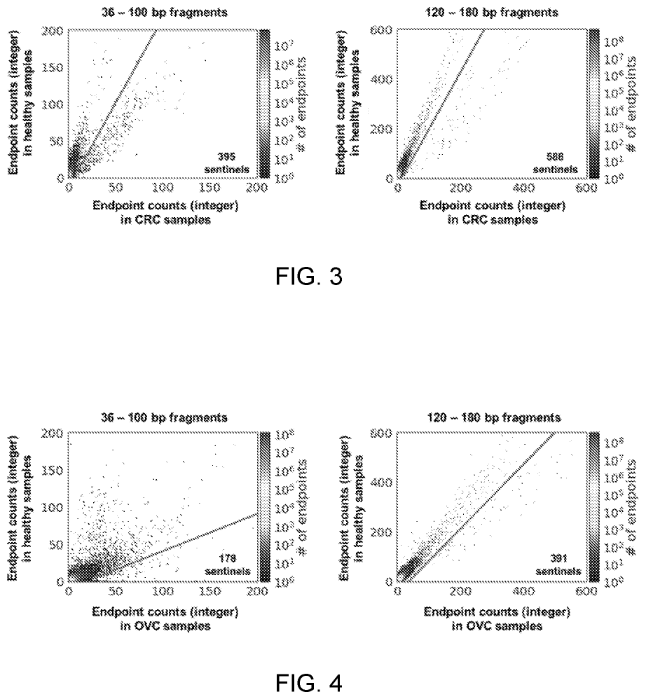

Under a null hypothesis that no fragment endpoints are associated with a disease state, points will tend to fall along a diagonal line, indicating that the genomic coordinates they represent are equally likely to be observed in either physiological state (See. FIG. 1). However, when a disease state is associated with fragment endpoints, the plot will evidence a set or sets of points that deviate from the expectation, forming one or more “thumbs” that extend, in a biased way, along one or more axes (See, FIG. 2). The thumbs consist of coordinates that are observed in a disease state and represent candidate sentinel endpoints.

In some embodiments, vectors are normalized to correct for differences in sequencing depth or coverage, fragment length distribution, local GC content, and chromosome number between the first physiological state, the second physiological state, and the subject. Normalization can be performed using standard techniques known to those skilled in the art.

### IX. Counting Fragment Endpoints at Genomic Locations and Linking Vectors

The number of fragment endpoints observed at each location are counted. In some embodiments, the number of fragment endpoints are counted with binary counts. Binary counts indicate endpoint counts such that each dataset contributes a one or a zero at each genomic coordinate under investigation. A value of one means that the coordinate was observed as an endpoint of at least one fragment in a sequenced library dataset. A value of zero, on the other hand, means that the coordinate was not observed as an endpoint in a sequenced library dataset. In a binary count, for example, the maximum count value for a given coordinate will be the number of libraries of a given physiological state.

In some embodiments, the number of fragment endpoints are counted with integer counts. Integer counts indicate endpoint counts such that each dataset contributes to the count at each coordinate based on the number of times that the coordinate appears as a fragment endpoint in that dataset. An integer count of three for a specific coordinate, for example, could mean that the coordinate was observed as an endpoint once in three different datasets, three times in a single dataset, or twice in one dataset and once in another.

Vectors are linked. In some embodiments, the vectors are linked by linking genomic locations between the vectors. In some embodiments, the vectors are linked by summing genomic locations between the vectors. In some embodiments, after fragment endpoint extraction, endpoints observed at each genomic location are counted within physiological states and across samples in at least one of two ways, as either binary counts or integer counts.

### X. Selecting Fragment Endpoints and Genomic Annotations

In some embodiments, the method further comprises filtering isolated cfDNA to retain cfDNA having a length between an upper bound and a lower bound. In some embodiments, the upper bound is 200, 190, 180, 170, 160, 150, 140, 130, 120, 110, 100, 90, 80, 70, 60, or 50 base pairs and the lower bound is 20, 25, 30, 35, 36, 40, 45, 50, 60, 70, 80, 90, 100, 110, or 120 base pairs. In some embodiments, only fragments falling within a specified length range, such as 36-100 base pairs, are retained. In some embodiments, filtering comprises gel electrophoresis and/or capillary electrophoresis.

In some embodiments, the method further comprises filtering sentinel endpoints based upon proximity to one or more genomic locations or one or more genomic annotations. In some embodiments, a subset of isolated cfDNA is targeted to a genomic location. In some embodiments, the genomic location comprises one or more genomic annotations. In some embodiments, the one or more genomic annotations comprises DNA-binding or DNA-contacting proteins.

Genomic annotations enrich genomic locations by providing functional information related to location in the genome. Once a genome is sequenced, it can be annotated to make sense of it. For DNA annotation, a previously unknown sequence representation of genetic material is enriched with information relating genomic position to intron-exon boundaries, regulatory sequences, repeats, gene names, and protein products. The National Center for Biomedical Ontology (www.bioontology.org) develops tools for annotation of database records based on the textual descriptions of those records.

in some embodiments, the one or more genomic annotations comprises or consists of transcription start sites. A transcription start site is the location where transcription starts at the 5′-end of a gene sequence. As the starting place for transcription, proteins involved in transcription may be expected to affect and influence fragment. endpoints, especially between one physiological state and another.

In some embodiments, the one or more genomic annotations comprises or consists of nucleosomes. Nucleosomes are known to be well positioned in relation to landmarks of gene regulation, for example transcriptional start sites and exon-intron boundaries.

### XI. Physiological States and Conditions

In some embodiments, cfDNA is isolated for the disease or physiological condition, at least one first physiological state, or at least one second, physiological state. The disease or physiological condition, at least one first physiological state, or at least one second physiological state comprise one or more healthy states or one or more disease states. In some embodiments, the one or more disease states comprise or consist of cancer, normal pregnancy, complications of pregnancy, myocardial infarction, inflammatory bowel disease, systemic autoimmune disease, localized autoimmune disease, allotransplantation with rejection, allotransplantation without rejection, stroke, and localized tissue damage.

In some embodiments, the disease or physiological condition, at least one first physiological state, or at least one second physiological state comprises or consists of cancer. In some embodiments, cancer comprises or consists of acute lymphoblastic leukemia; acute myeloid leukemia; acute myeloid leukemia; adrenocortical carcinoma; AIDS-Related cancers; anal cancer; astrocytomas; central nervous system cancers; basal cell carcinoma; bile duct cancer; bladder cancer; bone cancers; brain stem glioma; brain tumors; craniopharyngioma; ependymoblastoma; medulloblastoma; medulloepithelima; pineal parenchymal tumors; neuroectodermal tumors; breast cancer; bronchial tumors; Burkett's lymphoma; gastrointestinal cancers; cervical cancers; chronic lymphocytic leukemia; chronic myelogenous leukemia; chronic myeloproliferative disorders; colon cancer; colorectal cancer; cutaneous T-Cell lymphomas; endometrial cancers; esophageal cancers; Ewing cancers; extracranial germ cell tumors; eye cancers; retinoblastoma; gallbladder cancers; gastric cancers; gastrointestinal stromal tumor (GIST); ovarian cancers; hairy cell leukemia; head and neck cancer; heart cancer, hepatocellular cancers; Hodgkin's lymphoma; Kaposi's sarcoma; kidney cancers; lip and oral cavity cancers; liver cancers; lung cancers; non-small cell lung cancer; lymphoma; Waldenström macroglobulinemia; melanomas; mesothelioma; metastatic squamous neck cancers; mouth cancers; nasopharyngeal cancers; neuroblastoma; ovarian cancers; pancreatic cancer; penile cancers; pituitary tumors; rectal cancers; salivary gland cancers; squamous cell carcinomas; stomach cancers; throat cancers; thyroid cancers; and vaginal cancers. In some embodiments, cancer consists of colorectal cancer or ovarian cancer.

In some embodiments, the at least one first physiological state consists of a cancer at a first clinical stage (e.g., stage I) and the at least one second physiological state consists of a cancer at a second clinical stage (e.g., stage IV). In some embodiments, the first clinical stage consists of a cancer at stage 0, stage I, stage II, stage III, or stage IV. In some embodiments, the second clinical stage consists of a cancer at stage 0, stage I, stage II, stage or stage IV.

In some embodiments, the disease or physiological condition, at least one first physiological state, or at least one second physiological state comprises or consists of normal pregnancy Or complications of pregnancy. In some embodiments, the disease or physiological condition, at least one first physiological state, or at least one second physiological state comprises or consists of myocardial infarction or inflammatory bowel disease, in some embodiments, the disease or physiological condition, at least one first physiological state, or at least one second physiological state comprises or consists of allotransplantation with rejection and/or allotransplantation without rejection.

### XII. Sentinel Endpoints

Some embodiments provide for one or more sentinel endpoints. Some embodiments define a mathematical function to segregate distributions of quantities from the linked vectors into a first group and a second group, the first group comprising genomic coordinates with lesser difference to the mathematical function and the second group comprising genomic coordinates with greater difference to the mathematical function and identifying one or more sentinel endpoints as members of the second group.

In some embodiments, the greater difference of quantities comprises or consists of about 10%, about 20%, about 30%. about 40%, about 50%, about 60%, about 70%, about 80%, about 90%, or about 95% greater difference of quantities between the second group and the mathematical function. In some embodiments, the lesser difference of quantities to the mathematical function comprises or consists of about 10%, about 20%, about 30%, about 40%, about 50%, about 60%, about 70%, about 80%, about 90%, or about 95% lesser difference of quantities between the first group and the mathematical function.

In some embodiments, the mathematical function comprises or consists of a filter. To identify members of the second group as sentinel endpoints, a filter is applied to the vectors or to a histogram or scatterplot resulting front the vectors, such that points lying one side of a filter are identified as sentinel endpoints and points on the other side of the filter are not. For example, if there is a single “thumb” of candidate sentinel endpoints as illustrated in FIG. 2, a filter may be implemented as a diagonal line that slices through the base of the thumb, with. points to the right and below the line being identified as sentinel endpoints.

In some embodiments, the filter comprises or consists of a heuristic Heuristic filters may identify sentinel endpoints by attempting to separate one or more “thumbs” in a 2-d histogram or scatterplot from a main cloud of points. In some embodiments, the filter comprises or consists of as linear or quadratic step function.

In some embodiments, the filter comprises or consists of a statistical filter. Statistical filtering is a more formal filtering approach. In statistical filtering an underlying generative model gives rise to fragment endpoint counts in sequencing datasets as a first group, and deviations from the first group, in a second group comprising or consisting of sentinel endpoints, may be identified by calculating a test statistic and/or p-value. In some embodiments, a statistical filter flattens and deconvolves a 2-d histogram or scatterplot as a mixture of two or more distributions, a first group representing a null or background. distribution giving rise to fragment endpoints along a diagonal and a second group representing a distribution of sentinel endpoints.

In some embodiments, defining a mathematical function comprises of consists using a Z-score.

Sentinel endpoints provide evidence of biased fragmentation patterns of native chromatin that distinguish cell types, mark cell death pathways, and point to damage or proliferation of specific tissues. A given disease state may have zero, few, or many sentinel endpoints, and these sentinel endpoints may change in strength or number over the spectrum of disease severity or progression.

In some embodiments, each sentinel endpoint is assigned a score or weight to represent or quantify a degree of relation to a disease state. For example, a sentinel endpoint may be assigned a score or weight representing the degree to which the sentinel endpoint is pathognomonic. In some embodiments, the score comprises or consists of a function of the proximity of the sentinel endpoint from the filter.

In some embodiments, each sentinel endpoint is assigned a score or weight to represent or quantify a degree of relation to one or more genomic annotations. As an example, if a specific gene is marked by many sentinel endpoints in its vicinity, and another gene is marked by few sentinel endpoints, sentinel endpoints near the first gene may be weighted more highly. In some embodiments, the one or more genomic annotations comprises DNA-binding or DNA-contacting proteins. In some embodiments, the one or more genomic annotations comprises or consists of transcription start sites. In some embodiments, the one or more genomic annotations comprises or consists of nucleosomes. In some embodiments, the one or more genomic annotations comprise or consist of the promoter of a gene.

In some embodiments, each sentinel endpoint is assigned a score or weight to represent or quantify a degree of relation to other sentinel endpoints. For example, a score may be calculated by subtracting the number of times a sentinel endpoint appears in all libraries representing a first disease state from the number of times the sentinel endpoint appears in all libraries representing a second disease state. As another example, a score may be calculated by dividing the number of times a sentinel endpoint appears in all. libraries representing a first disease state by the number of times the sentinel endpoint appears in all libraries representing the second disease state.

XIII. Classification of Samples with Sentinel Endpoints

Some embodiments comprise or consist of diagnosing a disease or physiological condition in a human if the number of sentinel endpoints in the subject vector is above a threshold value. Once a number of sentinel endpoints is empirically determined, a disease or physiological condition can be diagnosed if the number of sentinel endpoints is above the threshold value. In some embodiments, the threshold value is around about 900. around about 950, around about 1000, around about 1050, around about 1100, or around about 1150. Some embodiments provide a threshold value as set forth in example 3.

One skilled in the art will understand, that the number sentinel endpoints may increase based upon the amount of sequencing (the number of reads or sequencing coverage) that is done. A lower number of sequencing reads may produce a lower threshold value and a higher number of sequencing reads may produce a higher threshold. In some embodiments, the threshold value is adjusted proportionately to number of the sequencing reads.

### XIV. Computer Systems

Some embodiments comprise a computer system programmed to implement the methods provided herein. The computer system includes a central processing unit (“CPU”). The computer system also includes memory or memory location, electronic storage unit, communication interface for communicating with other systems, and peripheral devices, such as cache, other memory, data storage, and/or electronic display adapters. The memory, storage unit, interface, and peripheral devices are in communication with the CPU through a communication bus, such as a motherboard.

The storage unit can be a data storage unit. The computer system can be operatively coupled to a computer network. The network can be the Internet, an intranet and/or extranet, or an intranet and/or extranet that is in communication with the Internet. The network in some cases is a telecommunication and/or data network. The network can include one or more computer servers, which can enable distributed computing, such as cloud computing.

The CPU can execute a sequence of instructions, which can be embodied in a program or software. The instructions may be stored in the memory. The instructions can be directed to the CPU.

The computer system can include or be in communication with an electronic display that comprises a user interface for providing a report, which may include a diagnosis of a subject or a therapeutic intervention for the subject. The report may be provided to a subject, a health care professional, a lab-worker, or other individual.

### XV. Diagnosis, Reports and Treatment

Some embodiments comprise providing a report, and recommending treatment for the disease or physiological condition. An electronic report with scores can be generated to indicate diagnosis or prognosis. If an electronic report indicates there is a treatable disease, the electronic report can prescribe a therapeutic regimen or a treatment plan. A diagnosis of a physiological condition may be made by a qualified healthcare practitioner based on the sentinel endpoints or based on sentinel endpoints in combination with one or another factors.

### EXAMPLES

### Example 1

Frozen human plasma specimens were obtained in 3×1 ml aliquots from 15 healthy donors and from five individuals with clinical diagnosis of CRC. The specimens were thawed on the benchtop to approximately room temperature. Each specimen was processed in one batch with the Qiagen Circulating Nucleic Acid kit as per the manufacturer's protocol. Briefly, each plasma sample was placed in a 50 ml conical and combined with 300 μl Proteinase K and 2.4 ml Buffer ACL (lysis buffer). The tubes were vortexed for 30 seconds, covered with parafilm, and placed in a 60° C. water bath for 30 minutes. After incubation, the tubes were placed, on the bench, and 5.4 mL of Buffer ACB (binding buffer) was added to each sample, followed by vortexing for 30 seconds. The tubes were then placed on ice for 10 minutes. The full volume of each tube was loaded into a spin column with tube extender in a Qiagen vacuum manifold. Each column was washed with. 600 μl ACW1, 750 μl ACW2, and 750 μl 100% ethanol. The columns were spun at 17000×g for three minutes and the flow through was discarded, The columns were dried at room temperature with the lids open for 10 minutes. 40 μl of buffer AVE (elution buffer) was added to each column and incubated at room temperature for 10 minutes to elute the DNA. The DNA was collected in Lo-Bind tubes (Eppendorf) by.centrifugation at 17000×g for 2 minutes. cfDN.A yield was quantified by a Qubit fluorometer (Invitrogen) using a dsDNA HS kit. The purified ciDNA samples were then stored at −20° C.

To prepare sequencing libraries, a maximum of 30 ng of cfDNA in 10 μl buffer AVE was used as input. The indexed libraries were constructed using the ThruPLEX Plasma-seq kit (Rubicon Genomics) as per the manufacturer's protocol, comprising a proprietary series of end-repair, adapter ligation, and amplification steps. Library amplification was monitored with real-time PCR to avoid overamplification. After amplification, the PCR products were cleaned with. AMPure beads (Beckman Coulter) and eluted in 20 μl of buffer EB. Library fragment size was determined by gel electrophoresis and library concentration was determined by Qubit using a dsDNA HS kit. Libraries were pooled and diluted for sequencing on an Illumina Novaseq instrument with an S4 flow cell.

Paired-end, 2×100 base pairs reads were generated for the pooled libraries. After sequencing, the resulting sequencing data was split by sample index. Adapters were trimmed using the software cutadapt. The trimmed reads were aligned to the human reference gcnomc (version hg38) with the software bwa.

The two genomic endpoints of each properly paired fragment having mapping quality of at least 60 were extracted using a custom software program. This process was performed separately for fragments having lengths between 36 and 100 base pairs (inclusive) and between 120 and 180 base pairs (inclusive). The frequency of each endpoint coordinate in healthy and CRC samples is displayed in the FIG. 4. A linear mathematical function (line) was used to segregate non-sentinels from sentinels; points below and to the right of the line were defined as sentinels. The function for the 120-1.80 base pairs fragment range had the equation: Y=2.3X−35 for X>=20; for the 36-100 base pairs fragment range, the mathematical function was Y=2.3X−15 for X>=10.395 sentinels were identified in the 36-100 base pairs fragment range, and 588 sentinels were identified in the 120-180 base pairs fragment range.

FIG. 2 depicts binary count visualization with a single “thumb.” Simulated endpoint frequencies for 60,000 genomic coordinates were tallied in binary fashion and plotted as a 2-d histogram, as in FIG. 1. As can be seen, a cloud of sentinel endpoints extends along the X-axis, representing genomic coordinates that are preferentially observed in cancer samples but not healthy samples. The solid diagonal line is drawn to indicate that there is an equal number of samples in the two sample sets. The dashed diagonal line represents one possible heuristic filter.

### Example 2

Frozen human plasma specimens were obtained in 1 ml aliquots from 15 healthy donors and from eight individuals with clinical diagnosis of CVC. The specimens were thawed on the benchtop to approximately room temperature. Each specimen was processed in one batch with the Qiagen Circulating Nucleic Acid kit as per the manufacturer's protocol. Briefly, each plasma sample was placed in a 15 ml conical and combined with 100 μl Proteinase K and 0.8 ml Buffer ACL (lysis buftr). The tubes were vortexed for 30 seconds, covered with parafilm, and placed in a 60° C. water bath for 30 minutes. After incubation, the tubes were placed on the bench, and 1.8 mL of Buffer ACB (binding buffer was added to each sample, followed by vorlexing for 30 seconds. The tubes were then placed on ice for 10 minutes. The fill volume of each tube was loaded into a spin column with tube extender in a Qiagen vacuum manifold. Each column was washed with 600 μl ACW1, 750 ∥l ACW2, and 750 μl 100% ethanol. The columns were spun at 17000×g for 3 minutes and the flow through was discarded. The columns were dried at room temperature with the lids open for 10 minutes. 40 μl of buffer AVE (elution buffer) was added to each column and incubated at room temperature for 10 minutes to elute the DNA. The DNA was collected in Lo-Bind tubes (Eppendorf) by centrifugation at 17000×g for 2 minutes. cfDNA yield was quantified by a Qubit fluorometer (Invitrogen) using a dsDNA HS kit. The purified cfDNA samples wore then stored at −20° C.

To prepare sequencing libraries, a maximum of 30 ng of cfDNA in 10 μl buffer AVE was used as input. The indexed libraries were constructed using the ThruPLEX Plasma-seq kit (Rubicon Genomics) as per the manufacturer's protocol, comprising a proprietary series of end-repair, adapter ligation, and amplification steps. Library amplification was monitored with real-time PCR to avoid overamplification. After amplification, the PCR products were cleaned with AMPure beads (Beckman. Coulter) and elated in 20 μl of buffer EB. Library fragment size was determined by gel electrophoresis and library concentration was determined by Qubit using a dsDNA HS kit. Libraries were pooled and diluted for sequencing on an Illumina Novaseq instrument with an S4 flow cell.

Paired-end, 2×100 base pairs reads were generated for the pooled libraries. After sequencing, the resulting sequencing data was split by sample index. Adapters were trimmed using the software cutadapt. The trimmed reads were aligned to the human reference genome (version hg38) with the software bwa.

The two genomic endpoints of each properly paired fragment having mapping quality of at least 60 were extracted using a custom software program. This process was performed separately for fragments having lengths between 36 and 100 base pairs (inclusive) and between 120 and 180 base pairs (inclusive). The frequency of each endpoint coordinate in healthy and OVC samples is displayed in the FIG. 4. A linear mathematical function (line) was used to segregate non-sentinels from sentinels; points below and to the right of the line were defined as sentinels. The function for the 120-180 base pairs fragment range had the equation: Y=1.3X−50 for X>=20; for the 36-100 base pairs fragment range, the mathematical function was Y=0.5X−10 for X>=25. 178 sentinels were identified in the 36-100 base pairs fragment range, and 391 sentinels were identified in the 120-180 base pairs fragment range.

### Example 3

Targeted sequencing data from 411 samples, including 76 healthy individuals and 335 individuals with a clinical diagnosis of CRC was obtained in 13AM format. All sequencing data was in obtained from illumina Hiseq or Illumina Nextseq instruments. Sequencing was performed in paired-end mode to obtain 2×150 cycle reads.

Reads were aligned to the human reference genome (version hg38) with the software bwa. The two genomic coordinates representing the alignment endpoints of each properly paired fragment having mapping quality of at least 60 were extracted using a custom software program. Only fragments having inferred lengths between 36 and 1.00 base pairs (inclusive) were considered. Genomic coordinates with calculated sequencing depth greater than 10-fold higher than the median depth were excluded from further analysis.

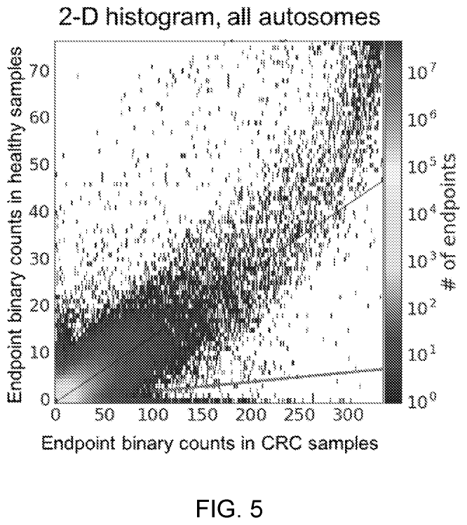

For each autosomal genomic coordinate, the number of sequencing datasets in which that genomic coordinate was observed at least once as a fragment endpoint was tallied (“binary counts”). This calculation was performed separately for healthy and CRC samples. The results are shown in FIG. 5. A filter (lower diagonal line) was applied to select the coordinates that show evidence of bias towards CRC samples. Endpoint coordinates falling below and to the right of this filter line were retained. In this example, 319 binary sentinel endpoint coordinates were retained.

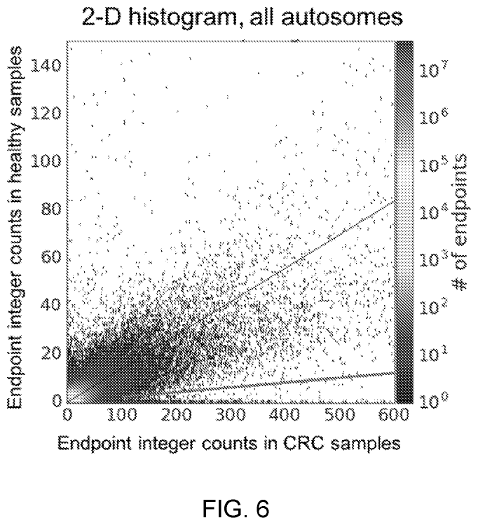

Separately, for each autosomal genomic coordinate, the number of fragments for which that coordinate appeared as an endpoint was tallied (“integer counts”). This calculation was performed separately for healthy and CRC samples. The results are shown in FIG. 6. A filter (lower diagonal line) was applied to select the coordinates that show evidence of bias towards CRC samples; the filter had the equation Y=0.02X for X>=100. Endpoint coordinates falling below and to the right of this filter line were labelled. sentinels and retained in the analysis. In this example, 1039 integer sentinel endpoint coordinates were retained.

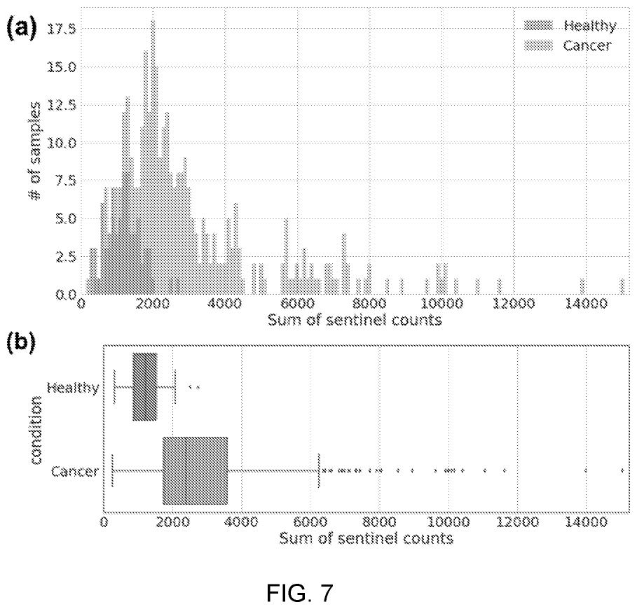

The number of sentinel endpoint observations was tallied at each of the set of 1039 integer sentinel endpoint coordinates. As expected, healthy samples evidenced a lesser number of sentinel endpoint observations, on average, than did CRC samples. The distributions for healthy and. CRC samples are shown in histogram form in FIG. 7A, and in boxplot form in FIG. 7B.

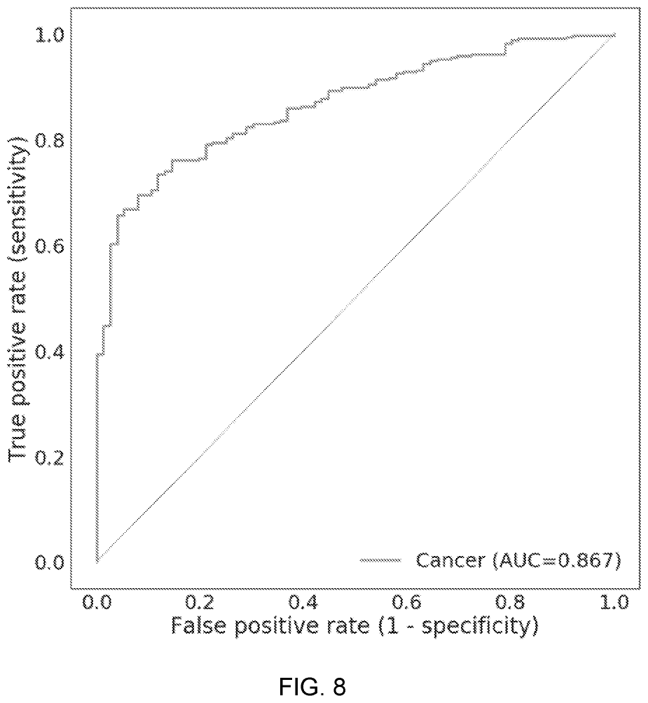

These two distributions were used as the basis for a binary classifier, to assign a label of “healthy” or “cancer” to each sample based on its number of sentinel endpoint. observations. The performance of this classifier is summarized in the receiver operating characteristic (ROC) curve shown in FIG. 8. The classifier yielded an area under the ROC curve (AUC) of 0.867.

AR publications and patent applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof.

1. A method of identifying one or more sentinel endpoints comprising:
a. isolating cell-free DNA (cfDNA) from biological sample(s) from one or more subjects with at least one first physiological state, the isolated. cfDNA comprising a first plurality of cfDNA fragments;
b. constructing at least one first sequencing library from the first plurality of cfDNA fragments;
c. sequencing first fragment endpoints of the first plurality of cfDNA fragments;
d. determining genomic locations of the first fragment endpoints within a reference genome for at least some of the first plurality of cfDNA fragments as a function of the sequences:
e. determining a first vector comprising the number of first fragment endpoints observed at each genomic location;
f. isolating cfDNA from biological sample(s) from one or more subjects with at least one second physiological state, the isolated cfDNA comprising a second plurality of cfDNA fragments;
g. constructing at least one second sequencing library from the second plurality of cfDNA fragments;
h. sequencing second fragment endpoints of the second plurality of cfDNA fragments;
i. determining genomic locations of the second fragment endpoints within the reference genome for at least some of the second plurality of cfDNA fragments as a function of the sequences;
j. determining a second vector comprising the number of second fragment endpoints observed at each genomic location;
k. linking the first vector and the second vector;
l. defining a mathematical function to segregate distributions of quantities from the linked vectors into a first group and a second group, the first group comprising genomic coordinates 1, with lesser difference to the mathematical function and the second group comprising genomic coordinates with greater difference to the mathematical function; and
m. identifying one or more sentinel endpoints as members of the second group.

2. The method of claim 1, further comprising diagnosing a disease or physiological condition in a subject in need thereof wherein the at least one first physiological state is a healthy state and the at least one second physiological state is a disease state, comprising:
a. isolating cfDN. from biological sample(s) from the subject, the isolated cfDNA comprising a subject plurality of cfDNA fragments;
b. constructing a subject sequencing library from the subject plurality of cfDNA fragments;
c. sequencing subject fragment endpoints of the subject plurality of cfDNA fragments;
d. determining genomic locations of at least some of the subject fragment endpoints within the reference genome as a function of the sequences;
e. determining a subject vector comprising the number of subject fragment endpoints observed at each genomic location; and
f. diagnosing the disease or physiological condition in the subject if the number of sentinel endpoints in the subject vector is above a threshold value.

3. The method of claim 1, wherein some of the isolated cfDNA are filtered to retain cfDNA having a length between an upper bound and a lower bound.

4. The method of claim 3, wherein the upper bound is 200, 190, 180, 170, 160, 150, 140, 130, 120, 110, 100, 90, 80, 70, 60, or 50 base pairs and the lower bound is 20, 25, 30, 35, 36, 40, 45, 50, 60, 70, 80, 90, 100, 110, or 120 base pairs.

5. The method of claim 1, wherein a subset of any of the isolated cfDNA is targeted to a genomic location.

6. The method of claim 5, wherein the genomic location comprises one or more genomic annotations.

7. The method of claim 6, wherein the one or more genomic annotations comprises or consists of transcription start sites (TSSs).

8. The method of claim 2, further comprising providing a report, with scores.

9. The method of claim 2, further comprising recommending treatment for the diagnosed disease or physiological condition in the subject

10. The method of claim 2, wherein the disease or physiological condition is selected from the group consisting of cancer, normal pregnancy, complications of pregnancy, myocardial infarction, inflammatory bowel disease, systemic autoimmune disease, localized autoimmune disease, allotransplantation with rejection, allotransplantation without rejection, stroke, and localized tissue damage.

11. The method of claim 10, wherein the cancer is colorectal cancer or ovarian cancer.

12. The method of claim 2, wherein the biological sample comprises or consists of whole blood, peripheral blood plasma, urine, or cerebral spinal fluid.

13. The method of claim 5, further comprising filtering sentinel endpoints based upon proximity to one or more genomic annotations.

14. The method of claim 13, wherein the one or more genomic annotations comprises or consists of transcription start sites (TSSs). 15 (Original) The method of claim 1, further comprising determining that a disease or physiological condition in a subject has an increased burden, severity, or clinical stage, wherein the at least one first physiological state is a disease state or physiological condition and the at least one second physiological state is the disease state or physiological condition with an increased burden, severity, or clinical stage, the method comprising:
a. isolating cfDNA from biological sample(s) from the subject, the isolated cfDNA comprising a subject plurality of cfDNA fragments;
b. constructing a subject sequencing library from the subject plurality of cfDNA fragments;
c. sequencing subject fragment endpoints of the subject plurality of cfDNA fragments;
d. determining genomic locations of the subject fragment endpoints within the reference genome for at least some of the subject plurality of cfDNA fragments as a function of the sequences;
e. determining a subj ect vector comprising the number of subj ect fragment endpoints observed at each genomic location;
f. comparing the subject vector to the sentinel endpoints;
g. identifying the burden, severity, or clinical stage of the disease or physiological condition as having an increased burden, severity, or clinical stage if the number of sentinel endpoints in the subj ect vector has more sentinel endpoints than a threshold value.
